(19) United States
(12) Patent Application Publication
(10) Pub. No.: US 2012/0022070 A1
(43) Pub. Date: Jan. 26, 2012

(54) Heat Shock Protein 90 Inhibitors, Methods Of Preparing Same, And Methods For Their Use

(75) Inventors: Jaeki Min, Marietta, GA (US); Pahk Thepchatri, Atlanta, GA (US); Yuhong Du, Atlanta, GA (US); Dennis C. Liotta, Atlanta, GA (US); Haian Fu, Decatur, GA (US)

(73) Assignee: EMORY UNIVERSITY, Atlanta, GA (US)

(21) Appl. No.: 13/122,798

(22) PCT Filed: Oct. 6, 2009
(86) PCT No.: PCT/US09/59677

§ 371 Date: Oct. 4, 2011

(30) Foreign Application Priority Data
Oct. 6, 2008 (US) 61/103172

(51) Int. Cl. A61K 31/5375; C07D 213/74; C07C 225/30; C07D 239/42; C07D 295/116; C07D 211/14; C07D 295/26; A61K 31/47; A61K 31/44; A61K 31/136; A61K 31/505; A61K 31/451; A61K 31/495; A61P 35/00; A61P 29/00; A61P 25/28; C07D 215/38

(52) U.S. Cl. 514/238.8; 546/171; 546/312; 564/429; 544/332; 544/173; 546/206; 544/395; 544/383; 514/311; 514/352; 514/657; 514/275; 514/319; 514/255.03

(57) ABSTRACT

Novel classes of molecular chaperone Heat shock protein 90 (Hsp90) inhibitors are disclosed. These compounds are useful in treating and preventing cancer and other Hsp90-related diseases and conditions, such as inflammation and neurodegenerative disorders. Methods of treating and preventing cancer and other Hsp90 related diseases and conditions are disclosed that include administering to the subject a therapeutically effective amount of an Hsp90 inhibitor. Methods of preparing the novel Hsp90 inhibitors are also provided.

### CROSS-REFERENCE TO RELATED APPLICATIONS

This application claims priority to U.S. Provisional Application No. 61/103,172, filed Oct. 6, 2008, which is incorporated herein by reference in its entirety.

### BACKGROUND

Cancer refers to a class of diseases that arises from the uncontrollable growth and division of normal cells. These malignant tumors are characterized by certain characteristics, including self-sufficiency in growth signaling, insensitivity to anti-growth signaling, evasion of apoptosis, sustained angiogenesis, tissue invasion and metastasis, and limitless potential to replicate (J. Biosci. 2007, 32, 517-530). Several of the key signaling proteins of cancer cells are maintained by heat shock protein 90 (Hsp90), which ranks amongst the most highly expressed cellular proteins. Hsp90 is a molecular chaperone with significant roles in maintaining transformation and in elevating the survival and growth potential of cancer cells. The biological role of Hsp90 is mediated by its ability to interact with client substrates such as Raf-1, Akt, Her2, cdk4, and Bcr-Abl. Hsp90 is required for the ATP-dependent refolding of denatured or “unfolded” proteins and for the conformational maturation of a subset of proteins involved in the response of cells to extracellular signals. Activation of signaling pathways mediated by these Hsp90 clients is necessary for cell proliferation, regulation of cell cycle progression, and apoptosis. Additionally, gain-of-function mutations responsible for transformation often require Hsp90 for maintenance of their folded, functionally active conformations. Oncogenic transformation often enhances the tumor cell's dependency on Hsp90 function.

Hsp90 also has a significant role in the progression of neurodegenerative diseases. Many neurodegenerative diseases, such as Alzheimer's and Parkinson's diseases, are characterized by misfolded and mutated proteins. These pathogenic forms of the proteins depend upon Hsp90 for conformational stability. In addition, neurodegenerative diseases often result from deviant signaling pathways which often rely upon Hsp90 for functioning. The inhibition of this chaperone is a potential mechanism of treating these diseases. Hsp90-interfering drugs represent a class of therapeutic agents that by selective inhibition of the chaperone could exhibit a broad range of anti-tumor activity by affecting multiple aspects of transformation regulated by Hsp90. In addition, these agents represent a potential class of drugs that promote the survival of neurons and open up a promising approach for the treatment of neuro degenerative diseases.

### SUMMARY

Novel methods and compositions for treating and preventing cancer and Hsp90 related diseases or conditions such as, for example, inflammation and neurodegenerative disorders, are provided. The methods comprise administering to a subject a therapeutically effective amount of Hsp90 inhibitor. For example, a class of Hsp90 inhibitors can comprise compounds of the following formula:

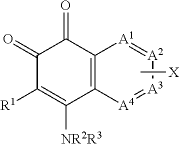

and includes pharmaceutically acceptable salts and prodrugs thereof. In this class of molecules, A¹, A², A³, and A⁴ are each independently selected from CH and N; X is hydrogen, an electron withdrawing group, or an electron donating group; R¹ is selected from hydrogen or halogen; and R² and R³ are each independently selected from hydrogen, substituted or unsubstituted C₁-C₁₂ cyanoalkyl, substituted or unsubstituted C₁-C₁₂ cyanohaloalkyl, substituted or unsubstituted C₂-C₁₂ cyanoalkenyl, substituted or unsubstituted C₂-C₁₂ cyanoalkynyl, substituted or unsubstituted cyanoaryl, substituted or unsubstituted cyanocycloalkyl, substituted or unsubstituted cyanoheteroalkyl, substituted or unsubstituted cyanoheterocycloalkyl, substituted or unsubstituted cyanoarylalkyl, substituted or unsubstituted cyanoheteroarylalkyl, substituted or unsubstituted cyanocycloalkylalkyl, substituted or unsubstituted cyanoheterocycloalkylalkyl, substituted or unsubstituted C₁-C₁₂ aminoalkyl, substituted or unsubstituted C₁-C₁₂ aminohaloalkyl, substituted or unsubstituted C₂-C₁₂ aminoalkenyl, substituted or unsubstituted C₂-C₁₂ aminoalkynyl, substituted or unsubstituted aminoaryl, substituted or unsubstituted aminocycloalkyl, substituted or unsubstituted aminoheteroalkyl, substituted or unsubstituted aminoheterocycloalkyl, substituted or unsubstituted aminoarylalkyl, substituted or unsubstituted aminoheteroarylalkyl, substituted or unsubstituted aminocycloalkylalkyl, and substituted or unsubstituted aminoheterocycloalkylalkyl. In some embodiments, if R² is hydrogen, R³ is not hydrogen or 5-[1-methylethyl)amino]pentyl]amino. In some embodiments, NR²R³ is

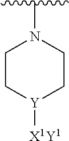

wherein X¹ is carbonyl or sulfonyl; Y is N or CZ, wherein Z is hydrogen, hydroxyl, C₁₋₁₂ alkyl, C₁₋₄ alkylaryl, or trifluoromethyl; and Y¹ is hydrogen, cyano, amino, halo, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl. In some embodiments, NR²R³ is:

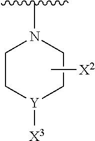

wherein X² is hydrogen, alkyl, or aryl; X³ is substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, unsubstituted aryl, or aryl substituted with halo, alkoxy, hydroxyl, carboxyl, nitro or trifluoromethyl; and Y is as defined above.

Also provided is a method of treating Hsp90 related diseases in a subject that includes selecting a subject with an Hsp90 related disease (e.g., cancer, a neurodegenerative disorder, or an inflammation causing disease) and administering to the subject a therapeutically effective amount of a compound of the following formula:

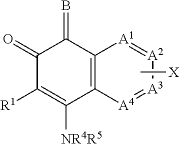

or pharmaceutically acceptable salts and prodrugs thereof. In this compound, A¹, A², A³, and A⁴ are each independently selected from CH and N; B is O or NTR⁶, wherein T is CH₂, O, or NH, and R⁶ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl; X is hydrogen, an electron-withdrawing group, or an electron-donating group; R¹ is hydrogen or halogen; and R⁴ and R⁵ are each independently selected from hydrogen, cyano, amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl. In some embodiments, NR⁴R⁵ is

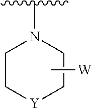

wherein W is hydrogen, an electron withdrawing group, or an electron donating group; and Y is CZR⁷, O, S, NR⁷, or NX¹—R⁷, wherein Z is hydrogen, hydroxyl, C₁₋₁₂ alkyl, C₁₋₄ alkylaryl, or trifluoromethyl; R⁷ is hydrogen, cyano, amino, substituted or unsubstituted C₁₋₁₂ alkyl, substituted or unsubstituted C₁₋₁₂haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl; and X¹ is carbonyl or sulfonyl.

Also provided is a method of preparing compounds for use as Hsp90 inhibitors. The method of preparing compounds wherein A¹, A², and A³ are CH and A⁴ is N comprises treating a substituted or unsubstituted 8-amino-5,6-dimethoxyquinoline with an alkyl or aryl halide in the presence of a base to produce a substituted or unsubstituted 8-alkylamino-5,6-dimethoxyquinoline or a substituted or unsubstituted 8-arylamino-5,6-dimethoxyquinoline; and treating a substituted or unsubstituted 8-alkylamino-5,6-dimethoxyquinoline or a substituted or unsubstituted 8-arylamino-5,6-dimethoxyquinoline with acid followed by a hydroxide to form a substituted or unsubstituted 8-alkylamino-5,6-quinolinedione or a substituted or unsubstituted 8-arylamino-5,6-quinolinedione. The method of preparing compounds wherein A¹, A², A³, and A⁴ are CH comprises treating 1,2-naphthoquinone-4-sulfonic acid sodium salt with an alkyl amine or aryl amine in the presence of a base to form a N-substituted 4-amino-1,2-naphthoquinone.

The details of one or more embodiments are set forth in the description below. Other features, objects, and advantages will be apparent from the description and from the claims.

### DETAILED DESCRIPTION

The term “comprising” and variations thereof as used herein are used synonymously with the term “including” and variations thereof and are open, non-limiting terms.

Novel classes of Hsp90 inhibitors are disclosed. These compounds are useful in treating, preventing, and/or ameliorating cancer and other Hsp90 associated diseases or conditions such as, for example, inflammation and neurodegenerative disorders. Specifically, pharmaceutically acceptable salts, prodrugs, and derivatives of 1,2-naphthoquinone and 5,6-quinolinedione compounds are provided. Methods of their synthesis and use in the treatment of Hsp90 associated conditions are also provided.

A method of treating Hsp90 related diseases in a subject includes selecting a subject with an Hsp90 related disease and administering to the subject a therapeutically effective amount of a compound represented by Formula I:

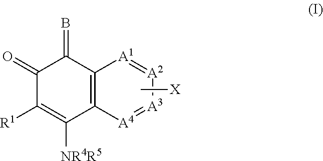

or pharmaceutically acceptable salts and prodrugs thereof.

In Formula I, A¹, A², A³, and A⁴ are each independently selected from CH and N.

Also, in Formula I, B is O or NTR⁶, wherein T is CH₂, O, or NH, and R⁶ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl.

Additionally, in Formula I, X is hydrogen, an electron-withdrawing group, or an electron-donating group. In some examples, X is hydrogen.

Also, in Formula I, R¹ is hydrogen or halogen.

Further, in Formula I, R⁴ and R⁵ are each independently selected from hydrogen, cyano, amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

The —NR⁴R⁵ group of Formula I can have, for example, the following Structure A1:

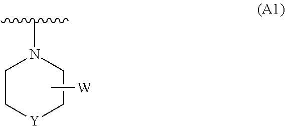

wherein W is hydrogen, an electron withdrawing group, or an electron donating group and Y is CZR⁷, O, S, NR⁷, or NX¹—R⁷, wherein Z is hydrogen or hydroxyl, X¹ is carbonyl or sulfonyl, and R⁷ is hydrogen, cyano, amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl. In some examples, W is selected from hydrogen, alkyl, alkoxyl, aryl, trifluoromethyl, amino, hydroxyl, halo, or nitro. In some examples, Y is NC₁-C₁₀ substituted or unsubstituted alkyl; NC₁-C₁₀ substituted or unsubstituted alkylaryl; NR⁹ wherein R⁹ is a substituted heteroaryl; NX¹—R⁷, wherein X¹ is sulfonyl; CZR⁷, wherein R⁷ is C₁-C₁₀ alkyl; or CZR⁷, wherein R⁷ is a substituted aryl.

In some examples of Formula I, R⁶ is substituted with a cyano group.

In other examples of Formula I, R⁶ is substituted with an amino group.

In some examples of Formula I, R⁴ and R⁵ are each independently selected from hydrogen, cyano, amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl. In these examples, at least one of R⁴ and R⁵ is substituted with cyano.

In some examples of Formula I, R⁴ and R⁵ are each independently selected from hydrogen, cyano, amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl. In these examples, at least one of R⁴ and R⁵ is substituted with amino.

In some examples of Formula I, X is hydrogen, alkyl, alkoxy, hydroxyl, halo, nitro, amino, trifluoromethyl, aryl, arylalkyl, or heteroaryl. In some examples, aryl, arylalkyl, and heteroaryl in X is selected from phenyl, benzyl, phenethyl, pyridinyl, and pyrimidinyl. The alkyl, aryl, arylalkyl or heteroaryl groups in X are optionally substituted with hydrogen, alkyl, alkoxy, trifluoromethyl, amino, hydroxyl, halo, or nitro.

In some examples of Formula I, A¹, A², and A³ are CH; A⁴ is N; B is O; and R¹ is hydrogen or chloride.

In other examples of Formula I, A¹, A², A³, and A⁴ are CH; B is O; and R¹ is hydrogen.

In some examples of Formula I, B is NTR⁶, wherein R⁶ is hydrogen, C₁-C₁₀ alkyl-V, wherein V is hydrogen, R⁸, —CO₂R⁸, —CO₂H, —CO₂NHR⁸, —NH₂, —NHC₁-C₁₀ alkyl, —NH-aryl, or N(CH₂CH₂)₂Q-R⁸, wherein R⁸ is hydrogen, cyano, amino, substituted or unsubstituted C₁₋₁₂ alkyl, substituted or unsubstituted C₁₋₁₂haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl; and Q is CH, O, or N.

In some embodiments, the Hsp90 inhibitors represented by Formula I include compounds represented by Formula II:

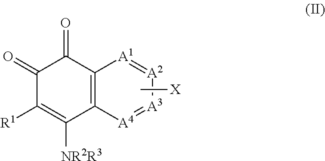

or pharmaceutically acceptable salts or prodrugs thereof.

In Formula II, A¹, A², A³, and A⁴ are each independently selected from CH and N.

Also, in Formula II, X is hydrogen, an electron-withdrawing group, or an electron-donating group.

Additionally, in Formula II, R¹ is hydrogen or halogen. In some examples, R¹ is hydrogen.

Further, in Formula II, R² and R³ are each independently selected from hydrogen, substituted or unsubstituted C₁-C₁₂ cyanoalkyl, substituted or unsubstituted C₁-C₁₂ cyanohaloalkyl, substituted or unsubstituted C₂-C₁₂ cyanoalkenyl, substituted or unsubstituted C₂-C₁₂ cyanoalkynyl, substituted or unsubstituted cyanoaryl, substituted or unsubstituted cyanocycloalkyl, substituted or unsubstituted cyanoheteroalkyl, substituted or unsubstituted cyanoheterocycloalkyl, substituted or unsubstituted cyanoarylalkyl, substituted or unsubstituted cyanoheteroarylalkyl, substituted or unsubstituted cyanocycloalkylalkyl, substituted or unsubstituted cyanoheterocycloalkylalkyl, substituted or unsubstituted C₁-C₁₂ aminoalkyl, substituted or unsubstituted C₁-C₁₂ aminohaloalkyl, substituted or unsubstituted C₂-C₁₂ aminoalkenyl, substituted or unsubstituted C₂-C₁₂ aminoalkynyl, substituted or unsubstituted aminoaryl, substituted or unsubstituted aminocycloalkyl, substituted or unsubstituted aminoheteroalkyl, substituted or unsubstituted aminoheterocycloalkyl, substituted or unsubstituted aminoarylalkyl, substituted or unsubstituted aminoheteroarylalkyl, substituted or unsubstituted aminocycloalkylalkyl, and substituted or unsubstituted aminoheterocycloalkylalkyl. In some examples, R² is hydrogen. In some examples, R³ is C₂-C₅ cyanoalkyl or C₂-C₅ aminoalkyl.

In some examples of Formula II, if R² is H, R³ is not H or 5-[1-methylethyl)amino]pentyl]amino.

The —NR²R³ group of Formula II can have, for example, the following Structure B1:

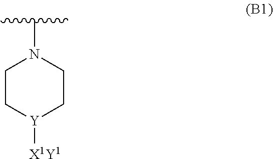

wherein X¹ is carbonyl or sulfonyl; Y is N or CZ, wherein Z is hydrogen, hydroxyl, C₁₋₁₂ alkyl, C₁₋₄alkylaryl, or trifluoromethyl; and Y¹ is hydrogen, cyano, amino, halo, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

The —NR²R³ group of Formula II can also have, for example, the following Structure B2:

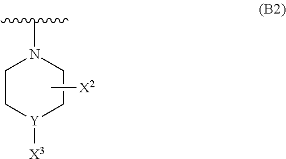

wherein X² is hydrogen, alkyl, or aryl; X³ is substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, unsubstituted aryl, or aryl substituted with halo, alkoxy, hydroxyl, carboxyl, nitro or trifluoromethyl; and Y is N or CZ.

The —NR²R³ group of Formula II can alternatively have, for example, the following structure B3:

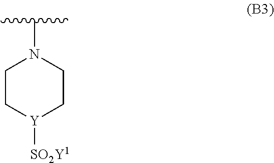

wherein A¹, A², A³ and A⁴ are CH; X is H; and Y¹ is substituted or unsubstituted aryl.

In one example, NR²R³ is selected from the following structures B4-B7:

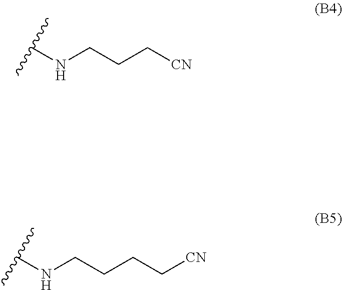

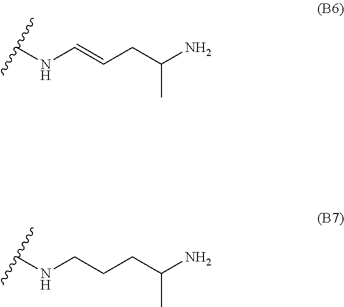

In one example, A¹, A², and A³ are CH and A⁴ is N; X is H; and R² and R³ are each independently selected from hydrogen, substituted or unsubstituted C₂-C₅ cyanoalkyl, substituted or unsubstituted C₂-C₅ cyanohaloalkyl, substituted or unsubstituted C₂-C₅ cyanoalkenyl, substituted or unsubstituted C₂-C₅ cyanoalkynyl, substituted or unsubstituted cyanoaryl, substituted or unsubstituted cyanocycloalkyl, substituted or unsubstituted cyanoheteroalkyl, substituted or unsubstituted cyanoheterocycloalkyl, substituted or unsubstituted cyanoarylalkyl, substituted or unsubstituted cyanoheteroarylalkyl, substituted or unsubstituted cyanocycloalkylalkyl, substituted or unsubstituted cyanoheterocycloalkylalkyl, substituted or unsubstituted C₂-C₅ aminoalkyl, substituted or unsubstituted C₂-C₅ aminohaloalkyl, substituted or unsubstituted C₂-C₅ aminoalkenyl, substituted or unsubstituted C₂-C₅ aminoalkynyl, substituted or unsubstituted aminoaryl, substituted or unsubstituted aminocycloalkyl, substituted or unsubstituted aminoheteroalkyl, substituted or unsubstituted aminoheterocycloalkyl, substituted or unsubstituted aminoarylalkyl, substituted or unsubstituted aminoheteroarylalkyl, substituted or unsubstituted aminocycloalkylalkyl, and substituted or unsubstituted aminoheterocycloalkylalkyl.

In another example, R² is hydrogen and R³ is substituted or unsubstituted C₂-C₅ cyanoalkyl, substituted or unsubstituted C₂-C₅ cyanohaloalkyl, substituted or unsubstituted C₂-C₅ cyanoalkenyl, substituted or unsubstituted C₂-C₅ cyanoalkynyl, substituted or unsubstituted cyanoaryl, substituted or unsubstituted cyanocycloalkyl, substituted or unsubstituted cyanoheteroalkyl, substituted or unsubstituted cyanoheterocycloalkyl, substituted or unsubstituted cyanoarylalkyl, substituted or unsubstituted cyanoheteroarylalkyl, substituted or unsubstituted cyanocycloalkylalkyl, substituted or unsubstituted cyanoheterocycloalkylalkyl, substituted or unsubstituted C₂-C₅ aminoalkyl, substituted or unsubstituted C₂-C₅ aminohaloalkyl, substituted or unsubstituted C₂-C₅ aminoalkenyl, substituted or unsubstituted C₂-C₅ aminoalkynyl, substituted or unsubstituted aminoaryl, substituted or unsubstituted aminocycloalkyl, substituted or unsubstituted aminoheteroalkyl, substituted or unsubstituted aminoheterocycloalkyl, substituted or unsubstituted aminoarylalkyl, substituted or unsubstituted aminoheteroarylalkyl, substituted or unsubstituted aminocycloalkylalkyl, or substituted or unsubstituted aminoheterocycloalkylalkyl, wherein the amino group in R³ is not substituted.

The X group can be, for example, hydrogen, alkyl, alkoxy, hydroxyl, halo, nitro, amino, trifluoromethyl, aryl, arylalkyl or heteroaryl, wherein the aryl, arylalkyl, and heteroaryl groups are selected from phenyl, benzyl, phenethyl, pyridinyl, and pyrimidinyl. The alkyl, alkoxy, aryl, arylalkyl, heteroaryl and amino groups in X can be independently substituted with hydrogen, alkyl, alkoxy, trifluoromethyl, amino, hydroxyl, halo, or nitro.

In some examples of Formula I or Formula II, the compound is a hydrochloride salt.

Examples of Hsp90 inhibitors represented by Formula I include compounds of Formula III and specific examples are provided below:

(III)

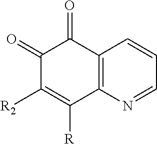

| Compound | R₁ | R₂ | Compound | NR1R2 | |
|---|---|---|---|---|---|
| 1 | 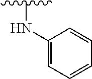 | H | 7 | 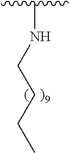 | Cl |
| 2 | 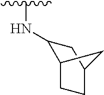 | Cl | 8 | 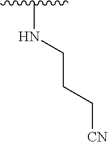 | H |
| 3 | 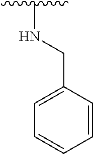 | Cl | 9 | 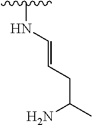 | H |
| 4 | 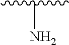 | H | 10 | 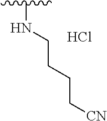 | H |
| 5 | 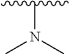 | Cl | 11 | 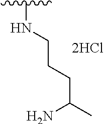 | H |
| 6 | 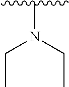 | Cl | | | |

Examples of Hsp90 inhibitors represented by Formula I include compounds of Formula IV and specific examples are provided below:

(IV)

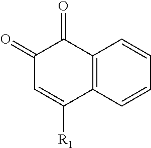

| Compound | R₁ |
|---|---|
| 12 | 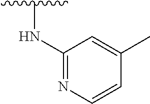 |
| 13 | 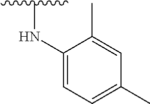 |
| 14 | 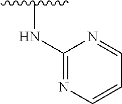 |
| 15 | 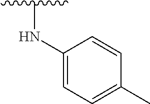 |
| 16 | 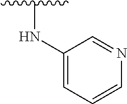 |
| 17 | 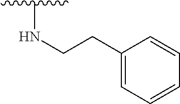 |
| 18 | 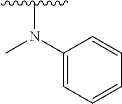 |
| 19 | 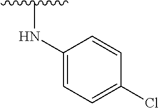 |
| 20 | 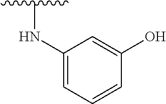 |
| 21 | 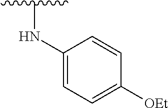 |
| 22 | 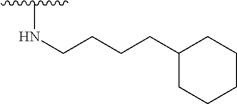 |
| 23 | 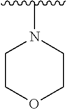 |
| 24 | 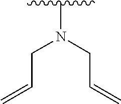 |
| 25 | 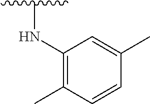 |
| 26 | 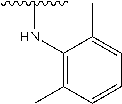 |
| 27 | 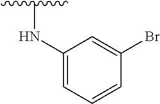 |
| 28 | 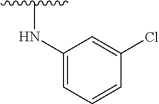 |
| 29 | 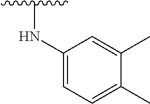 |
| 30 | 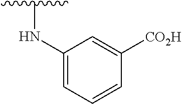 |
| 31 | 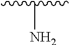 |
| 32 | 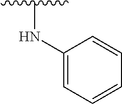 |
| 33 | 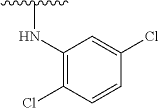 |
| 34 | 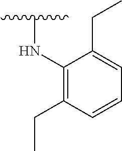 |
| 35 | 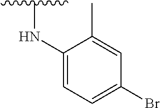 |
| 36 | 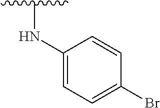 |
| 37 | 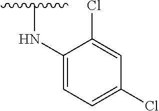 |
| 38 | 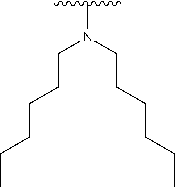 |
| 39 | 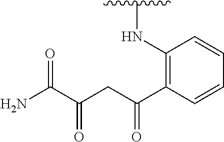 |
| 40 | 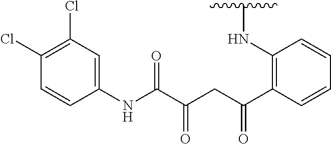 |
| 41 | 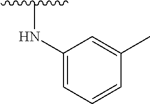 |
| 42 | 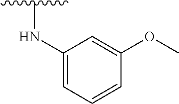 |
| 43 | 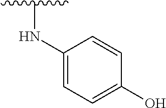 |
| 44 | 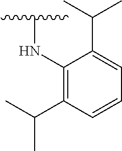 |
| 45 | 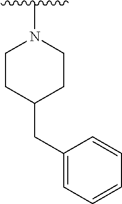 |
| 46 | 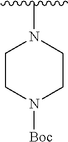 |
| 47 | 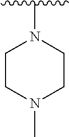 |
| 48 | 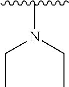 |
| 49 | 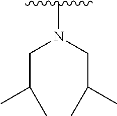 |
| 50 | 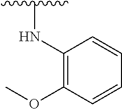 |
| 51 | 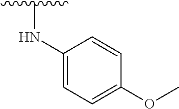 |
| 52 | 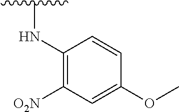 |
| 53 | 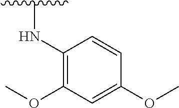 |
| 54 | 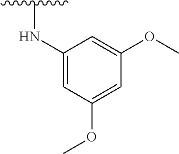 |
| 55 | 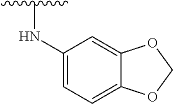 |
| 56 | 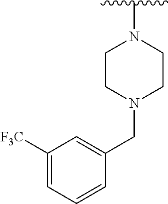 |
| 57 | 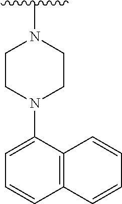 |
| 58 | 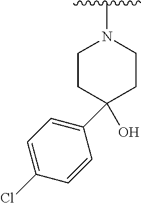 |
| 59 | 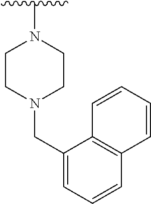 |
| 60 | 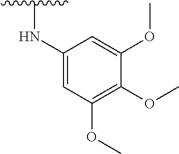 |
| 61 | 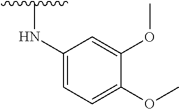 |
| 62 | 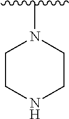 |
| 63 | 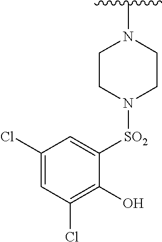 |
| 64 | 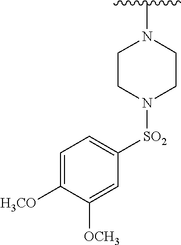 |
| 65 | 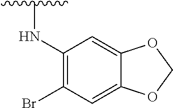 |
| 66 | 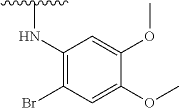 |
| 67 | 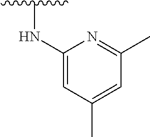 |
| 68 | 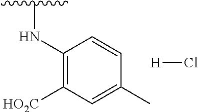 |

Variations on Formulas I, II, III, and IV include the addition, subtraction, or movement of the various constituents as described for each compound. Similarly, when one or more chiral centers is present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety. The synthesis and subsequent testing of various compounds as described for Formulas I and II to determine efficacy is contemplated.

As used herein, the terms “alkyl”, “alkenyl”, “alkynyl”, and “cycloalkyl” can include straight-chain and branched monovalent substituents. Examples include methyl, ethyl, isobutyl, cyclohexyl, cyclopentylethyl, 2-propenyl, 3-butynyl, and the like. “Heteroalkyl”, “heteroalkenyl”, “heteroalkynyl”, and “heterocycloalkyl” refer to compounds that are similar to “alkyl”, “alkenyl”, “alkynyl”, and “cycloalkyl” but that further contain a heteroatom such as O, S, N, or combinations thereof. The terms “cycloalkylalkyl” and “heterocycloalkylalkyl” refer to cycloalkyl and heterocycloalkyl groups that are bonded to alkyl. The term “aryl” can include monocyclic or fused bicyclic moieties such as phenyl or naphthyl and the term “heteroaryl” can include monocyclic or fused bicyclic ring systems containing one or more heteroatoms selected from O, S, and N. Heteroaryls can include, for example, 5-, 6-, 7-, and 8-membered rings. Thus, aryl and heteroaryl systems can include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl, and the like. Similarly, the terms “arylalkyl” and “heteroarylalkyl” refer to aryl and heteroaryl groups bonded to alkyl groups, including substituted or unsubstituted and saturated or unsaturated carbon chains. The term “substituted” indicates the main substituent has attached to it one or more additional components, such as, for example, OH, halogen, or one of the other substituents listed above. Substituted aryls can include, for example, monosubstituted, disubstituted, or trisubstituted aryls.

As used herein, the term “electron withdrawing group” refers to an atomic group that draws electrons from surrounding atomic groups by a resonance effect or an inductive effect more than a hydrogen atom would if it occupied the same position in the molecule. Electron withdrawing groups useful with the compounds and methods described herein include, for example, halogen (e.g. F, Br, Cl, or I), nitro, cyano, carboxyl, carbonyl, sulfonyl, trifluoromethyl, and trialkylaluminum.

As used herein, the term “electron donating group” refers to an atomic group that is capable of releasing electrons into surrounding atomic groups by a resonance effect or an inductive effect more than a hydrogen atom would if it occupied the same position in the molecule. Electron donating groups useful with the compounds and methods described herein include, for example, alkoxy, amino, aryl, and heteroaryl.

The 1,2-naphthoquinone compounds provided in Formula I can be prepared by reacting commercially available 1,2-naphthoquinone-4-sulfonic acid sodium salt (Aldrich; St. Louis, Mo.) with an alkyl or aryl amine at an equal molar ratio in the presence of base (e.g., K₂CO₃) in a water/ethanol solvent mixture. The reaction mixture can be diluted with water and cooled in an ice bath to obtain a solid that can be filtered and washed with a cold water/ethanol mixture. The desired product can then be dried in vacuo. The compounds 63 and 64 can be prepared, for example, by deprotecting compound 46 with 20% trifluoroacetic acid in dichloromethane to produce compound 62. Compound 62 can then be sulfonated by using substituted aryl sulfonyl chlorides in a mixture of pyridine and dichloromethane to give the desired products.

The 5,6-quinolinedione compounds described herein can be prepared using the process provided in Chem. Pharm. Bull., 1990, 38, 2841-2846, which is incorporated by reference herein in its entirety. For example, for the preparation of 8-amino-5,6-quinolinediones, 8-amino-5,6-dimethoxyquinoline can be reacted with an alkyl or aryl halide in the presence of triethylamine in an ethanol/n-butanol solvent mixture using a microwave initiator. The mixture can then be concentrated to give a dark oily residue which can be purified by chromatography to afford the desired products. The resulting N-substituted-5,6-dimethoxyquinoline can be converted to the corresponding 5,6-quinolinedione via oxidative demethylation with hydrobromic acid followed by neutralization using aqueous KOH, as described, for example in Kitahara et al. Chem. Pharm. Bull., 1990, 38, 2841-2846. The 5,6-quinolinedione can be immediately converted to the corresponding HCl salt form, if desired, by bubbling HCl gas in methanol.

The 5-(imino)quinolin-6-one compounds described herein can be prepared, for example, by reacting a 5,6-quinolinedione compound with a substituted amine and sodium acetate in solvent (e.g., methanol), as shown below.

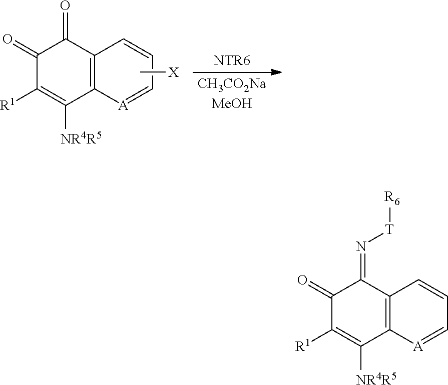

The compounds of Formula II, wherein A¹, A², and A³ are CH and A⁴ is N, can be prepared by treating a substituted or unsubstituted 8-amino-5,6-dimethoxyquinoline with an alkyl or aryl halide in the presence of a base to produce a substituted or unsubstituted 8-alkylamino-5,6-dimethoxyquinoline or a substituted or unsubstituted 8-arylamino-5,6-dimethoxyquinoline; and treating the substituted or unsubstituted 8-alkylamino-5,6-dimethoxyquinoline or the substituted or unsubstituted 8-arylamino-5,6-dimethoxyquinoline with acid followed by a hydroxide to form a substituted or unsubstituted 8-alkylamino-5,6-quinolinedione or a substituted or unsubstituted 8-arylamino-5,6-quinolinedione.

The compounds of Formula II wherein A¹, A², A³, and A⁴ are CH, can be prepared by treating 1,2-naphthoquinone-4-sulfonic acid sodium salt with an alkyl amine or aryl amine in the presence of base to form a N-substituted 4-amino-1,2-naphthoquinone.

Reactions to produce the compounds described herein can be carried out using the described or different solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The compounds described herein can be prepared using other methods known to one skilled in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

The compounds described herein or derivatives thereof can be provided in a pharmaceutical composition. Depending on the intended mode of administration, the pharmaceutical composition can be in the form of solid, semi-solid, or liquid dosage forms, such as, for example, tablets, suppositories, pills, capsules, powders, liquids, or suspensions, preferably in unit dosage form suitable for single administration of a precise dosage. The compositions can include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the compound described herein or derivatives thereof suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

The term pharmaceutically acceptable salt as used herein refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci., 1977, 66, 1, which is incorporated herein by reference in its entirety, at least, for compositions taught herein.)

The compounds described above or derivatives thereof are useful in treating cancer and other Hsp90 related diseases and conditions, such as inflammation and neurodegenerative disorders in humans, e.g., including pediatric and geriatric populations, and animals, e.g., veterinary applications. The methods described herein comprise administering to a subject a therapeutically effective amount of the compounds described herein or a pharmaceutically acceptable salt or prodrug thereof. Examples of Hsp90 related diseases include cancer and neurodegenerative diseases. The Hsp90 inhibitors described herein can also be used for the treatment of inflammation resulting from Hsp90 related diseases. As used herein the terms promoting, treating, and treatment includes prevention; delay in onset; diminution, eradication, or delay in exacerbation of one or more signs or symptoms after onset; and prevention of relapse.

The methods and compounds as described herein are useful for both prophylactic and therapeutic treatment. For prophylactic use, a therapeutically effective amount of the compounds described herein or derivatives thereof are administered to a subject prior to onset (e.g., before obvious signs of an Hsp90 related disease), during early onset (e.g., upon initial signs and symptoms of an Hsp90 related disease), or an established Hsp90 related disease. Prophylactic administration can occur for several days to years prior to the manifestation of symptoms of the Hsp90 related disease. Prophylactic administration can be used, for example, in the preventative treatment of subjects diagnosed with genetic Hsp90 related disease. Therapeutic treatment involves administering to a subject a therapeutically effective amount of the compounds described herein or derivatives thereof after an Hsp90 related disease is diagnosed.

Administration of compounds described herein or derivatives thereof can be carried out using therapeutically effective amounts of the compounds described herein or derivatives thereof for periods of time effective to treat Hsp90 related diseases. The effective amount of the compounds described herein or derivatives thereof may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

In these methods, an Hsp90 related disease, for example, can be further treated with one or more additional agents. The one or more additional agents and the compounds described herein or derivatives thereof can be administered in any order, including simultaneous administration, as well as temporally spaced order of up to several days apart. The methods may also include more than a single administration of the one or more additional agents and/or the compounds described herein or derivatives thereof. The administration of the one or more additional agents and the compounds described herein or derivatives thereof may be by the same or different routes and concurrently or sequentially.

The examples below are intended to further illustrate certain aspects of the methods and compounds described herein, and are not intended to limit the scope of the claims.

### EXAMPLES

### Example 1A

### Synthesis of 1,2-Naphthoquinones

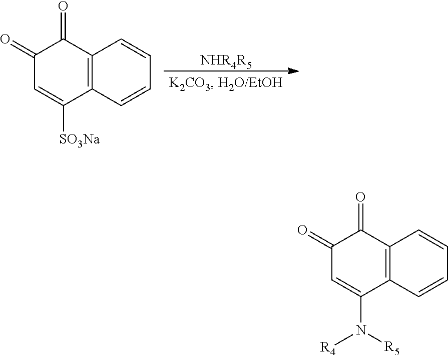

Commercially available 1,2-naphthoquinone-4-sulfonic acid sodium salt was reacted with the appropriate alkyl or aryl amine at an equal molar ratio in the presence of K₂CO₃ in a water/ethanol solvent mixture (Scheme A). The reaction mixture was diluted with water and cooled in an ice bath to obtain the solid that was filtered and washed with a cold water/ethanol mixture. The desired product was then dried in vacuo.

### Example 1B

### Synthesis of 1,2-Naphthoquinones

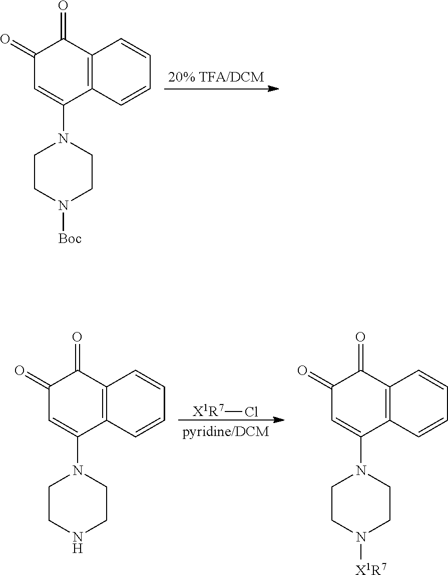

The compounds 63 and 64 were prepared by deprotecting compound 46 with 20% trifluoroacetic acid in dichloromethane to produce compound 62. Compound 62 was then sulfonated by using substituted aryl sulfonyl chlorides in a mixture of pyridine and dichloromethane.

### Example 2

### Synthesis of 5,6-Quinolinediones

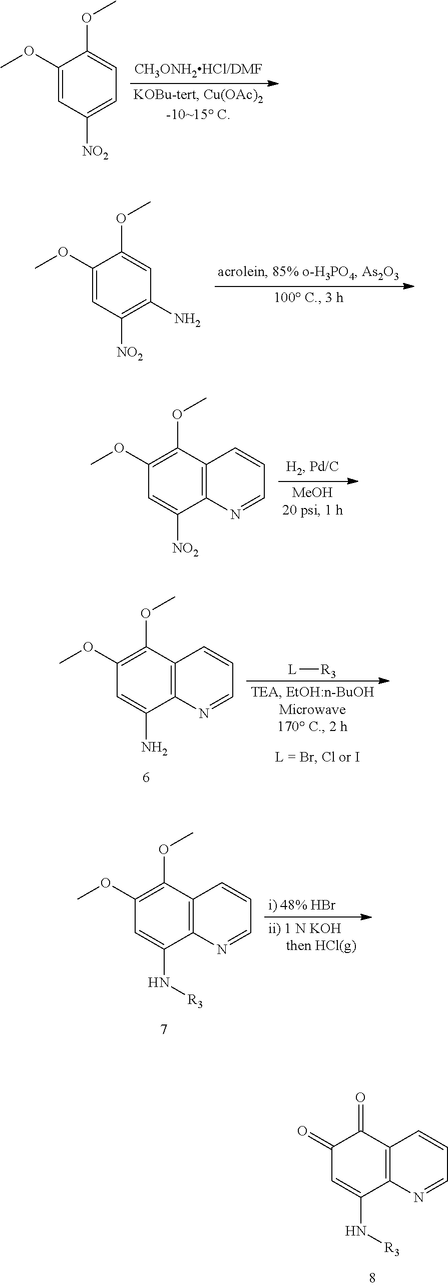

For the preparation of 8-amino-5,6-quinolinediones, 8-amino-5,6-dimethoxyquinoline was reacted with the appropriate alkyl or aryl halide in the presence of triethylamine under an ethanol/n-butanol (1/1) mixed solvent using a microwave initiator at 170° C. for 2.5 h. The mixture was concentrated to give a dark oily residue which was purified by chromatography to produce N-substituted 5,6-dimethoxyquinolines. The N-substituted 5,6-dimethoxyquinolines were converted to the corresponding 5,6-quinolinediones via oxidative demethylation with 48% hydrobromic acid followed by neutralization with aqueous KOH (Kitahara et al., Chem. Pharm. Bull., 1990, 38, 2841-2846). Some of the 5,6-quinolinediones were immediately converted to the HCl salt forms by bubbling HCl gas under a methanol solvent.

### Example 3

### Activity Characterization

The compounds were evaluated in several biological assays, including Hsp90 binding Fluorescence polarization (FP) assay, cell-based Her2 degradation assay by Western Blot (WB) or Cytoblot, and cell viability assay to test the growth inhibition effect of the compounds on cancer cells (Du, Moulick et al. J. Biomol. Screen, 2007, 12, 915-24). Assay protocols are briefly described below.

### Hsp90 Binding FP Assay:

There are several assays that have been developed to identify novel Hsp90 inhibitors based on the interaction of small molecules with recombinant Hsp90 protein, either yeast or human. To measure a therapeutically significant state of Hsp90, an FP assay that uses human cancer cell lysates instead of recombinant protein (Du, Moulick et al. J. Biomol. Screen, 2007, 12, 915-24) was utilized. It probes the interaction of small molecules with tumor-specific Hsp90 and may therefore lead to inhibitors that are not only selective for cancer cells but also specific for a particular malignancy.

The dose-response effect of the compounds was tested in Hsp90 binding FP competition assays to obtain the IC₅₀ of the compounds. The Hsp90 FP competition assay was performed in 384-well black plate using the small-cell lung carcinoma (SCLC) cell line NCI-N417 as a source of tumor specific Hsp90 and the Cy3B-labeled geldanamycin (GM-cy3B) as the FP ligand. The compound was dissolved in DMSO and added at several concentrations to the reaction buffer containing both GM-cy3B (5 nM) and NCI-N417 cell lysate (1 μg/well) in a final volume of 50 μL. Free GM-cy3B (5 nM) and bound GM-cy3B with NCI-N417 cell lysate (5 nM GM-cy3B and 1 μg/well of NCI-N417 cell lysate) were included as controls in each plate. After incubating at room temperature for 2 to 16 hours, the polarization values were measured and expressed as millipolarization (mP) units using Analyst HT plate reader (Molecular Devices). The competitive efficiency of the compounds were expressed as a percentage of control and calculated according to the following equation:

% of control=((mPc−mPf)/(mPb−mPf))×100

where mPc is the recorded mP from compound wells, mPf is the average recorded mP from GM-cy3B-only wells, and mPb is the average recorded mP from wells containing both GM-cy3B and NCI-N417 lysate. IC₅₀ values were determined using a nonlinear regression analysis as implemented in Prism 4.0 (Graphpad Software).

### Cell-Based Her2 Degradation Assay by Western Blot

Hsp90 uniquely stabilizes the Her2/Hsp90 association (Xu, Mimnaugh et al. J. Biol. Chem. 2001, 276, 3702-3708). Addition of Hsp90 inhibitors to cancer cells induces the proteasomal degradation of a subset of proteins involved in signal transduction such as Raf1 kinase, Akt and certain transmembrane tyrosine kinases such as Her2. Thus, Her2 degradation in cells is a functional read-out of Hsp90 inhibition. Correlation between Hsp90 binding and Her2 degradation in cancer cells is indicative of a selective biological effect in these cells via Hsp90.

A Western blot-based assay was used to measure the cellular level of Her2 protein in MCF-7 breast cancer cell lysates collected after 24 hours of compound treatment. MCF-7 cells were grown in RPMI-1640 medium supplemented with 10% fetal bovine serum (FBS). Then 2×10⁵ cells were seeded in 24 well plates in 500 μl PRMI1640 medium and allowed to attach overnight. The compounds were dissolved in DMSO, added to the wells, and incubated for 24 hours. The cells were washed using ice-cold phosphate-buffered saline (PBS) and lysed in 1% NP40 lysis buffer (10 mM HEPES, pH 7.4, 150 mM NaCl, 1% Nonident P-40, 5 mM Pyrophosphate, 5 mM NaF, 2 mM Orthovanadate, 10 ug/ml Aprotinin, 10 ug/ml Leupeptin, 1 mM PMSF). After boiling with 6×SDS sample buffer, the cell lysates were separated by SDS-PAGE and transferred to nitrocellulose membrane. The membranes were blocked with 5% milk in tris-buffered saline buffer. Her2 status was revealed by Western blotting with anti-Her2 antibody (Santa Cruz Biotechnology).

### Cell-Based Her2 Degradation Assay by Cytoblot

Cellular Her2 level after compound treatment was also monitored by microtiter-based Her2 degradation assay, Cytoblot assay (Huezo, Vilenchik et al. Chem. Biol., 2003, 10, 629-634). This is a cell-based assay that enables the quantitative analysis of intracellular levels of Her2 protein.

The human breast cancer cell lines SKBr3 were maintained in RPMI1640 medium supplemented with 10% FBS. 3000 cells in 100 μl of growth medium were plated per well in black, clear-bottom microtiter plates (Corning) and allowed to attach for at least 24 hours at 37° C. and 5% CO₂. Compounds at different doses or vehicle (DMSO) were carefully added to the wells and incubated for 24 hours. The cells were washed twice with ice-cold Tris buffer saline (TBS) containing 0.1% Tween 20 (TBST) and fixed with ice-cold methanol. After washing with TBST, the plate was incubated at RT for 1 hour with SuperBlock (Pierce 37535) and overnight at 4° C. with the anti-Her-2 antibody (Santa Cruz Biotechnology). The plate was then washed with TBST and incubated at RT for 2 hours with an anti-rabbit HRP-linked antibody (Sigma A0545). The chemiluminescent substrate solution (Pierce 38040) was added and the plate was read 5 minutes later in an Analyst HT plate reader (Molecular Devices). Luminescence readings resulting from compound-treated cells versus untreated cells (vehicle treated) were quantified and plotted against compound concentrations to give the IC₅₀ values (defined as concentration of compound required to degrade 50% of total Her2).

### Cancer Cell Growth Inhibition Assay

The effects of the compounds on cancer cell growth were determined using the CellTiter-Blue cell viability assay (Promega). CellTiter-Blue® Cell Viability is based on the ability of living cells to convert a redox dye (resazurin) into a fluorescent end product (resorufin). Nonviable cells rapidly lose metabolic capacity and thus do not generate a fluorescent signal.

The SKBR3 breast cancer cells were plated in 384-well microtiter plates (Costar) and allowed to attach overnight. After treatment with either compounds or vehicle (DMSO) for 96 hours, the cells were measured for their viability by CellTiter-Blue. Briefly, 10 μl of CelTiter-Blue were added to each well in 384-well plates and incubated at 37° C. for 4 hours. The fluorescence intensity (FI) was measured using the Analyst HT plate reader (Molecular Devices) with an excitation at 545 nm and emission at 595 nm. The IC₅₀ was calculated as the compound concentration that inhibits cell viability by 50% compared with vehicle control wells. The IC₅₀ values were determined as described above.

The IC₅₀ s of the compounds depicted in examples 1-44, 46-48, and 52 for the Hsp90 binding FP assay and Her2 degradation Western Blot (WB) assay are summarized (Table 1).

TABLE 1
Activity evaluation

| | | Her2 |
|---|---|---|
| | FP Assay | degradation |
| | IC₅₀ | WB IC50 |
| Compound | (μM) | (μM) |
| 1 | 0.09 | ~2.5 |
| 2 | 0.11 | ~2.5 |
| 3 | 0.14 | ~2.5 |
| 4 | 0.55 | >20 |
| 5 | 0.96 | >20 |
| 6 | 1.32 | >20 |
| 7 | 4.33 | ~5 |
| 8 | 0.31 | NA |
| 9 | 3.7 | >20 |
| 10 | 0.71 | ~10 |
| 11 | 0.45 | 2.5-5 |
| 12 | <0.39 | ~10 |
| 13 | 1.85 | <5 |
| 14 | 0.45 | 1-10 |
| 15 | 0.96 | ~10 |
| 16 | 0.67 | ~10 |
| 17 | 4 | ~10 |
| 18 | 0.39 | ~10 |
| 19 | 0.68 | <10 |
| 20 | 0.6 | ~10 |
| 21 | 1 | ~10 |
| 22 | inactive | >30 |
| 23 | 0.63 | ~10 |
| 24 | 2.19 | ~10 |
| 25 | 1.59 | >30 |
| 26 | 3.9 | >10 |
| 27 | — | >30 |
| 28 | — | >30 |
| 29 | 1.07 | <10 |
| 30 | 1.33 | >30 |
| 31 | 2.18 | 10-30 |
| 32 | 7.4 | <30 |
| 33 | 3.06 | >30 |
| 34 | 3.55 | >30 |
| 35 | 3 | >30 |
| 36 | 1.4 | ~30 |
| 37 | 4.7 | ~30 |
| 38 | 2.6 | ~30 |
| 39 | 4.2 | >30 |
| 40 | 10.5 | >30 |
| 41 | 1.06 | >=10 |
| 42 | 0.81 | ~10 |
| 43 | <0.39 | ~10 |
| 44 | 1.73 | >30 |
| 46 | 0.22 | 5~10 |
| 47 | 0.39 | 5~10 |
| 48 | 0.69 | 5~10 |
| 52 | 0.23 | >10 |

The IC₅₀ s of the compounds depicted in examples 45, 49-51, and 53-68 for the Hsp90 binding FP assay, Her2 degradation Western Blot (WB) and/or Cytoblot assay, and cell growth inhibitory assay (Skbr3 breast cancer cells) are summarized in Table 2.

TABLE 2
Activity evaluation reveals improved
potency of optimized compounds

| | | IC₅₀, Her2 | |
|---|---|---|---|
| | | degradation | |
| | FP Assay | WB or | IC₅₀, Growth |
| | IC₅₀ | Cytoblot(*) | inhibition |
| Examples | (μM) | (μM) | Assay (μM) |
| 62 | 1.55 | 22.67* | >20 |
| 63 | 1.31 | 26.09* | >20 |
| 64 | 0.57 | 29.14* | >20 |
| 68 | 0.58 | >30 | >20 |
| 51 | 0.07 | 1.25~2.5; 3.62* | 15.97 |
| 66 | 0.61 | 22.43* | 10.90 |
| 54 | 0.01 | 2.5~5; 4.17* | 7.46 |
| 65 | 0.53 | 15.33* | 3.66 |
| 61 | 0.39 | 4.83* | 3.18 |
| 55 | 0.04 | 2.5~5; 3.76* | 3.17 |
| 49 | 1.86 | 2.5~5; 18.64* | 2.75 |
| 60 | 0.18 | 8.58* | 2.42 |
| 53 | 0.13 | 2.5~5; 3.94* | 1.42 |
| 50 | 0.09 | 1.25~2.5; 3.82* | 1.24 |
| 45 | 0.23 | 1.25~2.5; 2.22* | 0.95 |
| 59 | 0.77 | 4.27* | 0.62 |
| 58 | 0.54 | 1.68* | 0.43 |
| 56 | 0.53 | 1.90* | 0.30 |
| 57 | 0.89 | 3.90* | 0.22 |
| 67 | 0.64 | 11.42* | 0.21 |

Several compounds reached nM potency for both the inhibition of Hsp90 activity and the inhibition of cancer cell growth (Table 2). It is expected that these potent compounds can be further developed as therapeutic agents for the treatment of a variety of cancers and other Hsp90-associated diseases.

A number of embodiments have been described. Nevertheless, it will be understood to one skilled in the art that various modifications may be made. Further, while only certain representative combinations of the formulations, methods, or products are disclosed herein are specifically described, other combinations of the method steps or combinations of elements of a composition or product are intended to fall within the scope of the appended claims. Thus a combination of steps, elements, or components may be explicitly mentioned herein; however, all other combinations of steps, elements, and components are included, even though not explicitly stated.

1. A compound of the following formula: 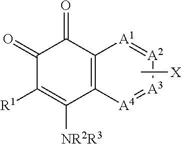
or a pharmaceutically acceptable salt or prodrug thereof, wherein:
A¹, A², A³, and A⁴ are each independently selected from CH and N;
X is hydrogen, an electron-withdrawing group, or an electron-donating group;
R¹ is hydrogen or halogen; and
R² and R³ are each independently selected from hydrogen, substituted or unsubstituted C₁-C₁₂ cyanoalkyl, substituted or unsubstituted C₁-C₁₂ cyanohaloalkyl, substituted or unsubstituted C₂-C₁₂ cyanoalkenyl, substituted or unsubstituted C₂-C₁₂ cyanoalkynyl, substituted or unsubstituted cyanoaryl, substituted or unsubstituted cyanocycloalkyl, substituted or unsubstituted cyanoheteroalkyl, substituted or unsubstituted cyanoheterocycloalkyl, substituted or unsubstituted cyanoarylalkyl, substituted or unsubstituted cyanoheteroarylalkyl, substituted or unsubstituted cyanocycloalkylalkyl, substituted or unsubstituted cyanoheterocycloalkylalkyl, substituted or unsubstituted C₁-C₁₂ aminoalkyl, substituted or unsubstituted C₁-C₁₂ aminohaloalkyl, substituted or unsubstituted C₂-C₁₂ aminoalkenyl, substituted or unsubstituted C₂-C₁₂ aminoalkynyl, substituted or unsubstituted aminoaryl, substituted or unsubstituted aminocycloalkyl, substituted or unsubstituted aminoheteroalkyl, substituted or unsubstituted aminoheterocycloalkyl, substituted or unsubstituted aminoarylalkyl, substituted or unsubstituted aminoheteroarylalkyl, substituted or unsubstituted aminocycloalkylalkyl, and substituted or unsubstituted aminoheterocycloalkylalkyl, wherein if R² is H, R³ is not H or 5-[1-methylethyl)amino]pentyl]amino; or
wherein NR²R³ is 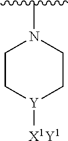
wherein X¹ is carbonyl or sulfonyl; Y is N or CZ, wherein Z is hydrogen, hydroxyl, C₁₋₁₂ alkyl, C₁₋₄ alkylaryl, or trifluoromethyl; and Y¹ is hydrogen, cyano, amino, halo, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl; or
wherein NR²R³ is: 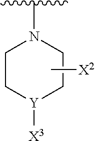
wherein X² is hydrogen, alkyl, or aryl; X³ is substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, unsubstituted aryl, or aryl substituted with halo, alkoxy, hydroxyl, carboxyl, nitro or trifluoromethyl; and Y is as defined above.

2. The compound of claim 1, wherein A¹, A², and A³ are CH and A⁴ is N.

3. The compound of claim 1, wherein X is H.

4. The compound of claim 1, wherein R² and R³ are each independently selected from hydrogen, substituted or unsubstituted C₂-C₅ cyanoalkyl, substituted or unsubstituted C₂-C₅ cyanohaloalkyl, substituted or unsubstituted C₂-C₅ cyanoalkenyl, substituted or unsubstituted C₂-C₅ cyanoalkynyl, substituted or unsubstituted cyanoaryl, substituted or unsubstituted cyanocycloalkyl, substituted or unsubstituted cyanoheteroalkyl, substituted or unsubstituted cyanoheterocycloalkyl, substituted or unsubstituted cyanoarylalkyl, substituted or unsubstituted cyanoheteroarylalkyl, substituted or unsubstituted cyanocycloalkylalkyl, substituted or unsubstituted cyanoheterocycloalkylalkyl, substituted or unsubstituted C₂-C₅ aminoalkyl, substituted or unsubstituted C₂-C₅ aminohaloalkyl, substituted or unsubstituted C₂-C₅ aminoalkenyl, substituted or unsubstituted C₂-C₅ aminoalkynyl, substituted or unsubstituted aminoaryl, substituted or unsubstituted aminocycloalkyl, substituted or unsubstituted aminoheteroalkyl, substituted or unsubstituted aminoheterocycloalkyl, substituted or unsubstituted aminoarylalkyl, substituted or unsubstituted aminoheteroarylalkyl, substituted or unsubstituted aminocycloalkylalkyl, and substituted or unsubstituted aminoheterocycloalkylalkyl.

5. The compound of claim 1, wherein R² is hydrogen and R³ is substituted or unsubstituted C₂-C₅ cyanoalkyl, substituted or unsubstituted C₂-C₅ cyanohaloalkyl, substituted or unsubstituted C₂-C₅ cyanoalkenyl, substituted or unsubstituted C₂-C₅ cyanoalkynyl, substituted or unsubstituted cyanoaryl, substituted or unsubstituted cyanocycloalkyl, substituted or unsubstituted cyanoheteroalkyl, substituted or unsubstituted cyanoheterocycloalkyl, substituted or unsubstituted cyanoarylalkyl, substituted or unsubstituted cyanoheteroarylalkyl, substituted or unsubstituted cyanocycloalkylalkyl, substituted or unsubstituted cyanoheterocycloalkylalkyl, substituted or unsubstituted C₂-C₅ aminoalkyl, substituted or unsubstituted C₂-C₅ aminohaloalkyl, substituted or unsubstituted C₂-C₅ aminoalkenyl, substituted or unsubstituted C₂-C₅ aminoalkynyl, substituted or unsubstituted aminoaryl, substituted or unsubstituted aminocycloalkyl, substituted or unsubstituted aminoheteroalkyl, substituted or unsubstituted aminoheterocycloalkyl, substituted or unsubstituted aminoarylalkyl, substituted or unsubstituted aminoheteroarylalkyl, substituted or unsubstituted aminocycloalkylalkyl, or substituted or unsubstituted aminoheterocycloalkylalkyl, wherein the amino group in R³ is not substituted.

6. The compound of claim 1, wherein R¹ is hydrogen.

7. The compound of claim 1, wherein R² is hydrogen.

8. The compound of claim 1, wherein R³ is C₂-C₅ cyanoalkyl.

9. The compound of claim 1, wherein R³ is C₂-C₅ aminoalkyl.

10. The compound of claim 1, wherein NR²R³ is

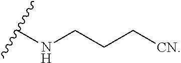

11. The compound of claim 1, wherein NR²R³ is

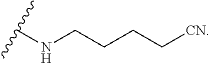

12. The compound of claim 1, wherein NR²R³ is

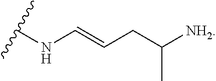

13. The compound of claim 1, wherein NR²R³ is

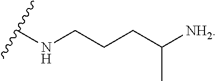

14-21. (canceled)

22. A composition comprising a compound of claim 1, and a pharmaceutically acceptable carrier.

23. A method of making the compounds described in claim 1, wherein A¹, A², and A³ are CH and A⁴ is N, comprising:
treating a substituted or unsubstituted 8-amino-5,6-dimethoxyquinoline with an alkyl or aryl halide in the presence of a base to produce a substituted or unsubstituted 8-alkylamino-5,6-dimethoxyquinoline or a substituted or unsubstituted 8-arylamino-5,6-dimethoxyquinoline; and
treating the substituted or unsubstituted 8-alkylamino-5,6-dimethoxyquinoline or the substituted or unsubstituted 8-arylamino-5,6-dimethoxyquinoline with acid followed by a hydroxide to form a substituted or unsubstituted 8-alkylamino-5,6-quinolinedione or a substituted or unsubstituted 8-arylamino-5,6-quinolinedione.

24. A method of preventing or treating cancer in a subject comprising administering to the subject a therapeutically effective amount of the compounds claim 1.

25. A method of preventing or treating Hsp90 related diseases in a subject, comprising administering to the subject a therapeutically effective amount of the compounds claim 1.

26. The method of claim 25, wherein the Hsp90 related disease causes inflammation.

27. The method of claim 25, wherein the Hsp90 related disease is a neurodegenerative disorder.

28-51. (canceled)
